(19) United States
(12) Patent Application Publication
(10) Pub. No.: US 2026/0321393 A1
(43) Pub. Date: Oct. 15, 2026

(54) METHOD AND SERVER FOR PROVIDING A DIAGNOSTIC SYSTEM ACCORDING TO A CONSULTATION PROCESS IN VIRTUAL REALITY

(71) Applicant: KOREA UNIVERSITY RESEARCH AND BUSINESS FOUNDATION, Seoul (KR)

(72) Inventors: Jae Bum JUNG, Seoul (KR); Kun Woo PARK, Seoul (KR); Kichun NAM, Seoul (KR); Changsu HAN, Seoul (KR); Serin JUNG, Incheon (KR); Sijeong WOO, Seoul (KR); Hyunseung CHOI, Incheon (KR)

(73) Assignee: KOREA UNIVERSITY RESEARCH AND BUSINESS FOUNDATION, Seoul (KR)

(21) Appl. No.: 19/572,426

(22) Filed: Mar. 19, 2026

(30) Foreign Application Priority Data
Mar. 19, 2025 (KR) 10-2025-0035464

(51) Int. Cl. G16H 20/70; G06F 3/01; G16H 10/20; G16H 40/67

(52) U.S. Cl. CPC G16H 20/70; G06F 3/011; G16H 10/20; G16H 40/67

(57) ABSTRACT

An operation method performed by a server operated by a processor according to an embodiment may comprise: an operation of acquiring basic information related to a client; a counseling center operation of providing a virtual reality space where the client may conduct psychological counseling on the basis of the basic information, allowing the client to conduct the psychological counseling, and recommending a first digital therapeutic among a plurality of digital therapeutics including an alleviation program for alleviating psychological risks of the client by analyzing a result of the psychological counseling; an information center operation of extracting customized information on the client on the basis of at least one among the result of the psychological counseling and the first digital therapeutic, and providing medical information including the extracted customized information; and an enhancement center operation of allowing the client to perform at least one among the first digital therapeutic and a second digital therapeutic selected by the client among the plurality of digital therapeutics, recording a result of performing at least one among the first digital therapeutic and the second digital therapeutic, and changing and storing the customized information on the basis of the recorded result of performing the digital therapeutic.

### TECHNICAL FIELD

The present invention relates to a method and a server for providing a diagnostic system according to a counseling process in virtual reality, and more particularly, to a method and a server for providing a system for personalized counseling including counseling, enhancement, and information provision processes performed in a counseling center in virtual reality such as a metaverse.

Meanwhile, this application is supported by the national research and development project described below.

Project ID: R2415881.

Ministry Name: Ministry of Science and ICT.

Title of Project Management (Specialized) Organization: Korea Radio Promotion Association.

Title of Research Business: 2024 Next-generation technology-leading metaverse project.

Title of Research Project: Development of digital healthcare and mind care platform based on AI metaverse.

Title of Project Implementing Organization: Korea Cloud Co., Ltd.

Research Period: May 1, 2025 to Dec. 31, 2025.

### BACKGROUND ART

In modern society, as diverse social relationships increase, the stress suffered by individuals is also increasing. Accordingly, psychological counseling is conducted to measure individual stress, identify its causes and severity, and provide solutions appropriate thereto.

Meanwhile, due to the infectious diseases such as COVID-19, the cases of processing conventional face-to-face works in a non-face-to-face non-contact manner in daily life are increasing recently, and accordingly, interest in non-face-to-face services such as online counseling is also increasing in the case of psychological counseling conducted one-on-one between people.

As examples of the non-face-to-face services, various types of non-face-to-face counseling services, such as counseling conducted in virtual reality, as well as video counseling and chat counseling conducted through the Internet, may be considered. There are some research insisting that non-face-to-face counseling has an effect practically equivalent to face-to-face counseling.

For example, according to “Gollings, E. K., & Paxton, S. J. (2006). Comparison of internet and face-to-face delivery of a group body image and disordered eating intervention for women: A pilot study. Eating Disorders: The Journal of Treatment & Prevention, 14, 1-15.”, as a result of conducting face-to-face group counseling and non-face-to-face group counseling on eating disorders for 6 months, only the face-to-face counseling is effective in the initial stage, but after 6 months later, symptom reduction of a similar level is observed from the eating disorders.

In addition, according to “Spence, S. H., Holmes, J. M., March, S., & Lipp, O. V. (2006). The Feasibility and Outcome of Clinic Plus Internet Delivery of Cognitive Behavior Therapy for Childhood Anxiety. Journal of Consulting and Clinical Psychology, 74(3), 614-621”, as a result of comparing a face-to-face treatment group and a treatment group who has conducted both the treatment through the Internet and face-to-face treatment to relieve anxiety disorder, the two groups show an effect of high-level improvement in the primary anxiety symptoms immediately after the treatment (65% vs. 56%) and in the long-term treatment (12 months, 89. 5% vs. 73.9%).

In addition, according to “Cohen, G. E., & Kerr, B. A. (1999). Computer mediated counseling: An empirical study of a new mental health treatment. Computers in human services, 15(4), 13-26”, as a result of confirming counseling results targeting male college students who have conducted counseling for the purpose of relieving anxiety, both the face-to-face counseling and the Internet counseling show the same effect when asking questions about anxiety during counseling, and there is no difference between the two.

In addition, according to “Germain, V., Marchand, A., Bouchard, S., Guay, S., & Drouin, M. (2010). Assessment of the therapeutic alliance in face-to-face or videoconference treatment for posttraumatic stress disorder. Cyberpsychology, Behavior and Social Networking, 13, 29-35”, as a result of conducting counseling once a week to prove the effectiveness of face-to-face counseling and video chat counseling groups for the participants with trauma, both the two groups show a significant reduction in the severity and frequency of the PTSD symptom, and the difference in the effectiveness between the groups is not significant.

In this way, even the non-face-to-face counseling may obtain a counseling effect of a level practically equivalent to that of face-to-face counseling.

However, conventional online psychological counseling service is an agency service that conducts tests simply in a way of conducting questionnaire, notifies the test subject of a test result, recommend specialized institutions when additional counseling or treatment is needed, and helps the test subject in making an appointment. However, a technique of automatically matching a client with an appropriate counselor or helping the client to receive psychological counseling more conveniently is insufficient.

In addition, since a system that properly provides follow-up management (e.g., utilization of digital therapeutics and therapeutic candidate groups) does not exist even when non-face-to-face treatment is conducted, and there is no system that provides medical information in relation to medical management for treatment conducted by patients or guardians, it is difficult to make follow-up medical management for the patients and guardians.

### PRIOR ART DOCUMENT

### Patent Document

Korean Patent Registration No. 10-2410322.

### DISCLOSURE OF INVENTION

### Technical Problem

Therefore, the present invention has been made in view of the above problems, and it is an object of the present invention to provide a technique of constructing a total solution platform server that provides comprehensive psychological treatment services, from the process of identifying psychological risk factors of a client on the basis of various information related to the client, to the enhancement process of allowing the client and a counselor to meet and conduct psychological service with each other in a non-contact manner and alleviating the psychological risk factors of the client, beyond the structure of conventional agency services that simply conduct tests in a way of conducting questionnaire and recommend specialized institutions.

In addition, another object of the present invention is to provide a technique of constructing a service providing platform server that systematically combines and supports a series of processes, including the procedures of questionnaire and counseling required for a user in virtual reality, counseling customized for a client, provision of digital treatment contents, association with external institutions, and case closures.

In addition, another object of the present invention is to present specific implementation examples of a counseling center, an enhancement center, and an information center included in the server.

Meanwhile, the technical problems of the present invention are not limited to the technical problems mentioned above, and unmentioned other technical problems can be clearly understood by those skilled in the art from the following description.

### Technical Solution

To accomplish the above objects, according to one aspect of the present invention, there is provided an operation method performed by a server operated by a processor, the method comprising: an operation of acquiring basic information related to a client; a counseling center operation of providing a virtual reality space where the client may conduct psychological counseling on the basis of the basic information, allowing the client to conduct the psychological counseling, and recommending a first digital therapeutic among a plurality of digital therapeutics including an alleviation program for alleviating psychological risks of the client by analyzing a result of the psychological counseling; an information center operation of extracting customized information on the client on the basis of at least one among the result of the psychological counseling and the first digital therapeutic, and providing medical information including the extracted customized information; and an enhancement center operation of allowing the client to perform at least one among the first digital therapeutic and a second digital therapeutic selected by the client among the plurality of digital therapeutics, recording a result of performing at least one among the first digital therapeutic and the second digital therapeutic, and changing and storing the customized information on the basis of the recorded result of performing the digital therapeutic.

In addition, the information center operation may further include an operation of performing a cognitive and emotional test on the basis of at least one among questionnaire information, behavioral information, and biometric information, wherein the questionnaire information may include information on a result of answering a predetermined questionnaire by the client, the behavioral information may include information on a response time and an error rate measured by the client by executing a predetermined measurement program, and the biometric information may include information on nonverbal elements, psychological index elements, and biometric signals of the client.

In addition, the operation method may further comprise, after the operation of acquiring basic information and before the counseling center operation, an operation of determining whether a result of the cognitive and emotional test previously performed by the counselor exists, wherein on the basis of the determining operation, the counseling center operation is performed when a result of the cognitive and emotional test exists, and the counseling center operation is performed subsequently after the cognitive and emotional test is performed when a result of the cognitive and emotional test does not exist.

In addition, the information center operation may perform an operation of allowing the counselor to perform the cognitive and emotional test after the counseling center operation, and the medical information further may include information on a result of the cognitive and emotional test.

In addition, the first digital therapeutic among the plurality of digital therapeutics including the alleviation program may include at least one among a counselor-recommended digital therapeutic recommended by the counselor by connecting the client to one of a plurality of counselors in the virtual reality space and performing psychological counseling by the counselor; and an AI-recommended digital therapeutic recommended by artificial intelligence on the basis of information acquired in the psychological counseling process performed by the counselor.

In addition, the medical information may further include academic information updated through medical seminars.

In addition, the academic information may be independently updated whenever the medical seminars are held or at predetermined intervals.

In addition, when the academic information is updated, the customized information of each client may be collectively changed on the basis of the updated academic information.

In addition, the customized information may further include information related to precautions needed for supporting the client, which is provided to a guardian of the client.

In addition, the enhancement center operation may further include an operation of providing a digital therapeutic for an element related to the client, among elements including at least one among post-traumatic stress disorder, personality disorder, sexual disorder, eating disorder, sleep disorder, adjustment disorder, and impulse control disorder, as the second digital therapeutic.

In addition, the enhancement center operation may further include an operation of providing a digital therapeutic for an element related to the client, among elements including at least one among musculoskeletal system, dry eye, tinnitus, chronic fatigue, decreased vision, and chest pain, as the second digital therapeutic.

In addition, the enhancement center operation may further include an operation of providing a digital therapeutic for an element related to the client, among elements including at least one among stroke, smoking, obesity, alcoholism, migraine, and cancer, as the second digital therapeutic.

According to another aspect of the present invention, there is provided a server comprising: a memory including instructions; and a processor that performs a predetermined operation based on the instructions, wherein the operation of the processor includes: an operation of acquiring basic information related to a client; a counseling center operation of providing a virtual reality space where the client may conduct psychological counseling on the basis of the basic information, allowing the client to conduct the psychological counseling, and recommending a first digital therapeutic among a plurality of digital therapeutics including an alleviation program for alleviating psychological risks of the client by analyzing a result of the psychological counseling; an information center operation of extracting customized information on the client on the basis of at least one among the result of the psychological counseling and the first digital therapeutic, and providing medical information including the extracted customized information; and an enhancement center operation of allowing the client to perform at least one among the first digital therapeutic and a second digital therapeutic selected by the client among the plurality of digital therapeutics, recording a result of performing at least one among the first digital therapeutic and the second digital therapeutic, and changing and storing the customized information on the basis of the recorded result of performing the digital therapeutic.

### Advantageous Effects

The present invention may allow a client and a counselor to conduct psychological service with each other in a non-contact manner by utilizing a virtual reality space such as a metaverse space, and provide a comprehensive psychological treatment platform construction technique, from the process of identifying psychological risk factors of the client on the basis of questionnaire/behavioral/biometric information related to the client, to the enhancement process of alleviating the psychological risk factors of the client by conducting counseling.

In addition, according to an embodiment of the present invention, through the construction of a one-stop counseling process system utilizing virtual reality, a user may be provided with multifaceted effects at once, such as diagnosis, symptom relief, and understanding of his/her state, by enhancing the cognitive function according to his/her state, medical knowledge related to him/herself, and medical enhancement activities.

In addition, according to an embodiment of the present invention, the system may allow a client to have cognitive and emotional digital treatment, as well as virtual remote counseling, on the platform, provide customized medical information to the client and the guardian of the client, and provide a one-stop customized counseling and treatment effect as appropriate treatment methods based on the medical information are continuously fed back to each unit.

Meanwhile, the effects of the present invention are not limited to those mentioned above, and unmentioned other technical effects will be clearly understood by those skilled in the art from the following descriptions.

### BRIEF DESCRIPTION OF THE DRAWINGS

FIG. 1 is a view showing the configuration of a diagnostic system according to a counseling process in virtual reality according to an embodiment.

FIG. 2 is a view showing the configuration of a diagnostic system providing server according to a counseling process in virtual reality according to an embodiment.

FIG. 3 is an exemplary view showing the configuration of a counseling center included in a diagnostic system providing server according to a counseling process in virtual reality according to an embodiment.

FIG. 4 is an exemplary view showing the configuration of an enhancement center included in a diagnostic system providing server according to a counseling process in virtual reality according to an embodiment.

FIG. 5 is an exemplary view showing the configuration of an information center included in a diagnostic system providing server according to a counseling process in virtual reality according to an embodiment.

FIG. 6 is a flowchart illustrating the steps of the operation performed by a diagnostic system providing server according to a counseling process in virtual reality according to an embodiment.

FIG. 7 is an exemplary view showing a survey tool for deriving a survey result based on questionnaire information of a client according to an embodiment.

FIGS. 8 and 9 are exemplary views showing a measurement program that perform psychological risk assessment of a client on the basis of behavioral information of the client according to an embodiment.

FIG. 10 is an exemplary view showing a result of performing a previously stored psychological risk assessment in response to a result of executing a measurement program according to an embodiment.

FIG. 11 is an exemplary view showing a neural network model trained to perform psychological risk assessment of a client on the basis of nonverbal elements of the client according to an embodiment.

FIG. 12 is an exemplary view showing a neural network model trained to perform psychological risk assessment on the basis of psychological index elements of a client according to an embodiment.

FIG. 13 is an exemplary view showing a metaspace provided by a server to connect a client and a counselor according to an embodiment.

FIG. 14 is an exemplary view showing a client profile according to an embodiment.

FIGS. 15 and 16 are exemplary view showing a game-type alleviation program that helps alleviate psychological risk factors of a client according to an embodiment.

FIG. 17 is an exemplary view showing an interactive avatar alleviation program that helps alleviate psychological risk factors of a client according to an embodiment.

### BEST MODE FOR CARRYING OUT THE INVENTION

Details of the objects and technical configurations of the present invention and operational effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are only examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software configurations that perform the same function.

In addition, the expressions including certain components are expressions of “open type” and only refer to existence of corresponding components, and should not be construed as excluding additional components.

Furthermore, when a certain component is referred to as being “connected” or “coupled” to another component, it may be directly connected or coupled to another component, but it should be understood that other components may exist in between.

Hereinafter, various embodiments of the present invention will be described with reference to the accompanying drawings. However, it should be understood that this is not intended to limit the present invention to specific embodiments, but to include various modifications, equivalents, and/or alternatives of the embodiments of the present invention.

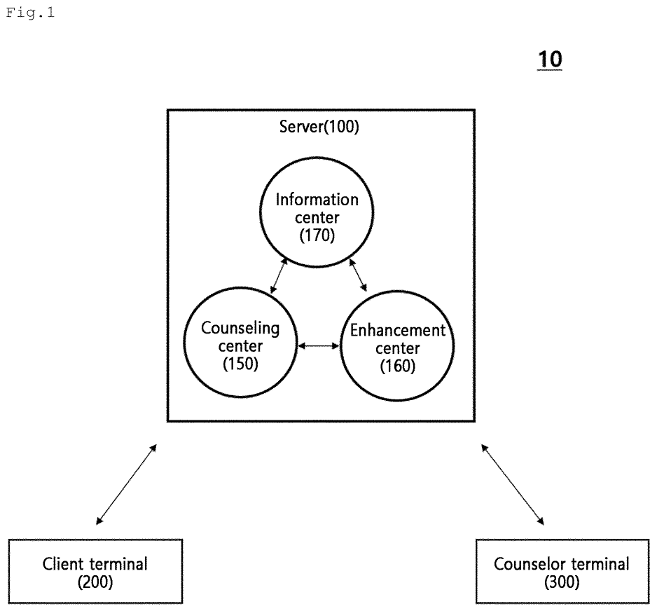

FIG. 1 is a view showing the configuration of a diagnostic system 10 according to a counseling process in virtual reality according to an embodiment (hereinafter, referred to as a ‘system 10’).

Referring to FIG. 1, the system 10 according to an embodiment may include a diagnostic system providing server 100 (hereinafter, referred to as a ‘server 100’) according to a counseling process in virtual reality, a client terminal 200, and a counselor terminal 300.

The server 100 is a computing device that provides platform service related to psychological counseling according to a counseling process. The server 100 may receive an access request from the client terminal 200, and provide a virtual reality space where the client of the client terminal 200 may conduct psychological counseling. The client of the client terminal 200 may conduct psychological counseling with the counselor of the counselor terminal 300. The server 100 may perform counseling center operations, information center operations, and enhancement center operations, which will be described below in more detail, for the psychological counseling on the client terminal 200.

The client terminal 200 is a terminal used by a client who desires to be provided with psychological service through the platform. The client may connect to the server 100 through the client terminal 200 and use the platform service provided by the server 100.

The counselor terminal 300 is a terminal used by a counselor who provides psychological service to the client through the platform. The counselor may connect to the server 100 through the counselor terminal 300 to provide counseling services to the client.

Referring to FIG. 1, in the counseling system performed in a virtual space according to an embodiment of the present invention, a client may connect to virtual reality (e.g., metaverse) by connecting to his or her client terminal 200 for disease treatment. The client may make an appointment and conduct counseling after selecting a topic that requires counseling. At this point, details of performing cognitive and emotional tasks or results of performing digital therapeutics may be retrieved and utilized for counseling. Psychological counseling may be conducted on the basis of interactions among the counseling center 150, the enhancement center 160, and the information center 170.

Basic psychological counseling may be conducted through the counseling center 150. When the psychological counseling is completed, a prescription for medication or digital therapeutics may be recommended by the counselor and/or artificial intelligence connected through the counselor terminal 300. The enhancement center 160 may preferentially provide digital therapeutics recommended through the counseling results, among various digital therapeutics. After the recommended digital therapeutics are completed, other digital therapeutics may be provided to be conducted. The information center 170 is a place where various information databases needed for the client are constructed, and it may implement a function of performing a cognitive and emotional test of the client and store results of performing the cognitive and emotional test. In addition, the information center 170 may store information customized for each client and latest academic information acquired from medical seminars for the client. The client and counselor may also acquire predetermined information from the information center 170 as needed.

The server 100, the client terminal 200, and the counselor terminal 300 according to an embodiment of the present invention may be implemented in various types of devices capable of transmitting and receiving information through a network. For example, the server 100, the client terminal 200, and the counselor terminal 300 may include portable communication devices (e.g., smartphones), computer devices, portable multimedia devices, laptop computers, tablet PCs, and the like.

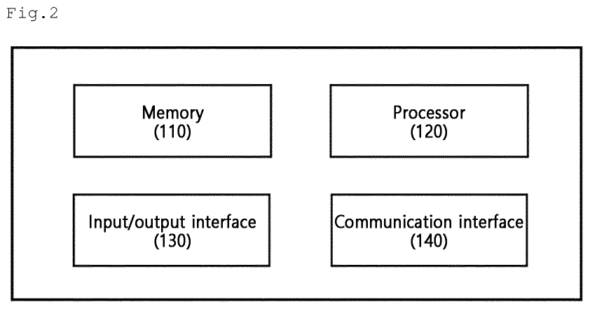

Hereinafter, components included in the server 100 according to an embodiment of the present invention and the operation of each component will be described with reference to FIG. 2.

FIG. 2 is a view showing the configuration of the server 100 according to an embodiment.

Referring to FIG. 2, the server 100 according to an embodiment may each include a memory 110, a processor 120, an input/output interface 130, and a communication interface 140.

The memory 110 may store data acquired from external devices or data generated by itself. The memory 110 may store instructions that may perform the operation of the processor 120. In addition, although the memory 110 may refer to a main memory device implemented as random access memory (RAM), it is not limited thereto. For example, the memory 110 is a concept encompassing a predetermined storage medium capable of temporarily or non-temporarily storing the instructions, and the storage medium may include RAM, memory buffer, hard drive, database, erasable programmable read-only memory (EPROM), electrically erasable read-only memory (EEPROM), read-only memory (ROM), and the like, and/or any combination thereof.

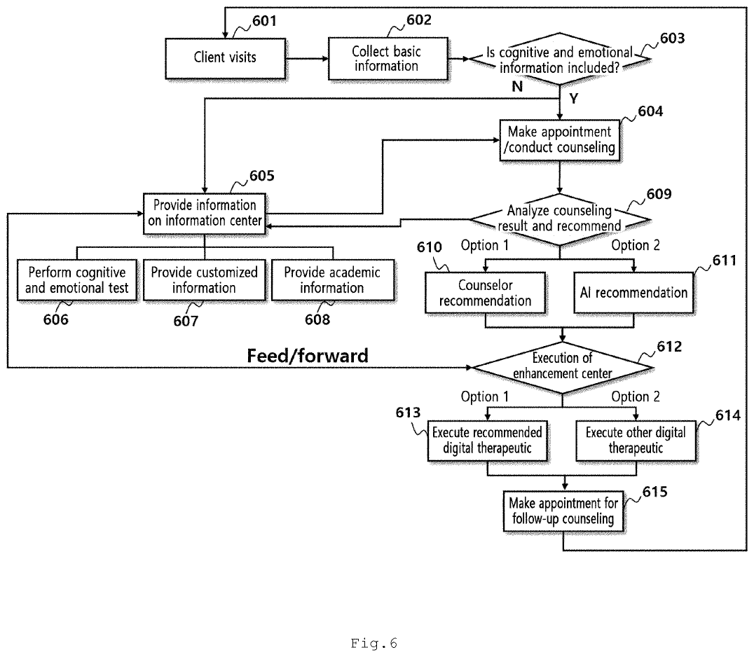

The processor 120 is a computing device that controls the overall operation. The processor 120 may execute instructions stored in the memory 110. The operation of the server 100 in FIG. 6 described below according to an embodiment of this document may be understood as an operation performed by the processor 120.

The input/output interface 130 may include a hardware interface or a software interface for inputting or outputting information.

The communication interface 140 allows to transmit and receive information through a communication network. To this end, the communication interface 140 may include a wireless communication module or a wired communication module.

The server 100 may be implemented in various types of apparatuses that can perform an operation through the processor 120 and transmit and receive information through a network. For example, although the apparatus may be implemented as a server, a computer apparatus, a portable communication apparatus, a smart phone, a portable multimedia apparatus, a laptop computer, a tablet PC, or the like, it is not limited to these examples.

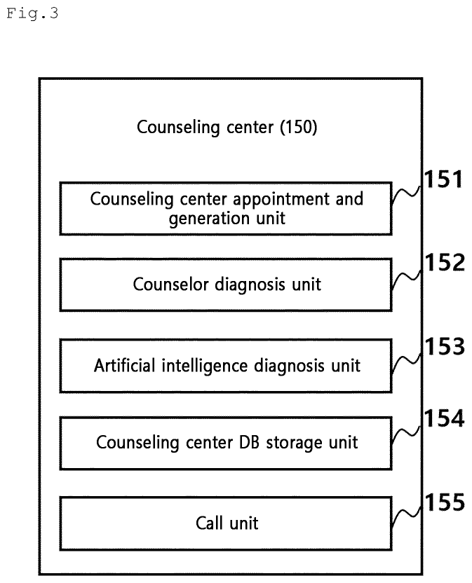

The exemplary embodiment shown in FIG. 2 is a view separately showing the components of the server 100 according to an embodiment of the present invention on the basis of the hardware configuration. However, FIGS. 3 to 5 are views separately showing components for performing the operations of the server 100 according to an embodiment of the present invention on the basis of logical/functional criteria. Terms such as the counseling center 150, the enhancement center 160, and the information center 170, which are logical/functional units in FIGS. 3 to 5, may also be distinguished as terms such as modules, units, or the like, and these logical/functional components may be distinguished as a software component, a hardware component, or a component integrating the software and hardware components. For example, the counseling center 150, the enhancement center 160, and the information center 170 may share the same memory 110, processor 120, input/output interface 130, communication interface 140, and the like as a means for implementing and executing them, or they may be distinguished and separately use a memory 110, a processor 120, an input/output interface 130, a communication interface 140, and the like of the same type, and it is natural to those skilled in the art that some of the components may share the same hardware component while the others may use different hardware components.

Hereinafter, an exemplary configuration of the counseling center 150, the enhancement center 160, and the information center 170 included in the diagnostic system providing server according to a counseling process in virtual reality according to an embodiment of the present invention will be described with reference to FIGS. 3 to 5.

FIG. 3 is an exemplary view showing the configuration of the counseling center 150 included in a diagnostic system providing server according to a counseling process in virtual reality according to an embodiment. As shown in FIG. 3, the counseling center 150 may include a counseling center appointment and generation unit 151, a counselor diagnosis unit 152, an artificial intelligence diagnosis unit 153, a counseling center DB storage unit 154, and a call unit 155.

The counseling center appointment and generation unit 151 may connect a client to one of a plurality of counselors to conduct psychological counseling. The counseling center appointment and generation unit 151 may create a space for conducting counseling by selecting a desired time, topic, counseling method, and counselor in response to the request of the client. For example, the space for conducting counseling may be a virtual reality space. The virtual reality may mean a world where objects or phenomena are shown to exist actually. Digitally constructed virtual reality spaces are useful to be utilized in a non-face-to-face manner as they are easy for the client to immerse in and allow communication with a counselor in various ways. An example of the virtual reality may include the metaverse.

The counselor diagnosis unit 152 may provide a counselor-recommended digital therapeutic recommended by the counselor as a first digital therapeutic. For example, after psychological counseling between the client and the counselor is completed through the counseling center appointment and generation unit 151, an operation of recommending a digital therapeutic may be implemented by the counselor on the basis of existing data of the client and counseling opinions of the counselor. The digital therapeutic may also be recommended and provided together with a prescription.

The artificial intelligence diagnosis unit 153 may implement an operation of recommending an artificial intelligence-recommended digital therapeutic as a first digital therapeutic on the basis of information acquired in the psychological counseling process conducted by the counselor. For example, the artificial intelligence diagnosis unit 153 may recommend a predetermined digital therapeutic on the basis of information such as counseling records, including conversation between the counselor and the client.

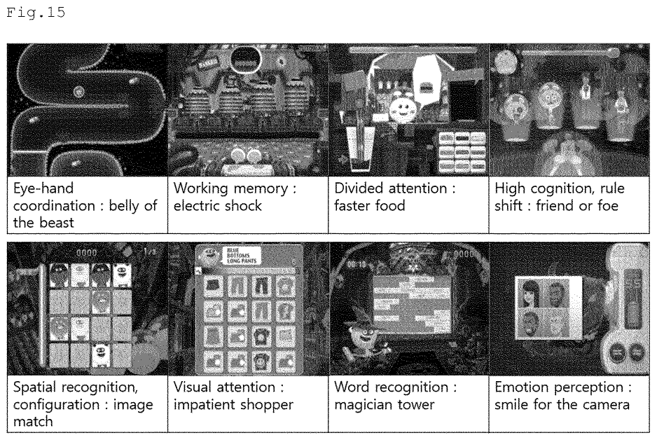

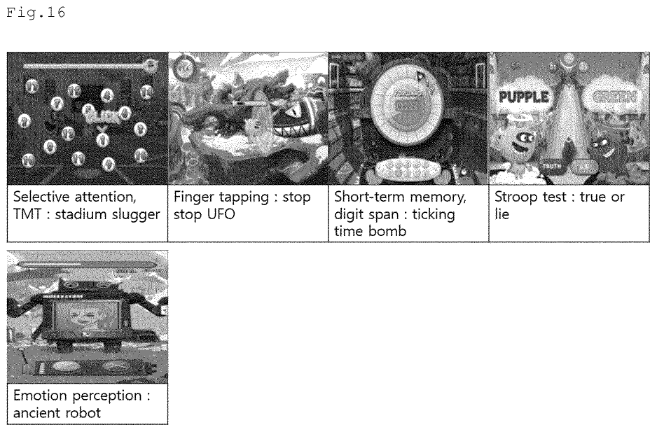

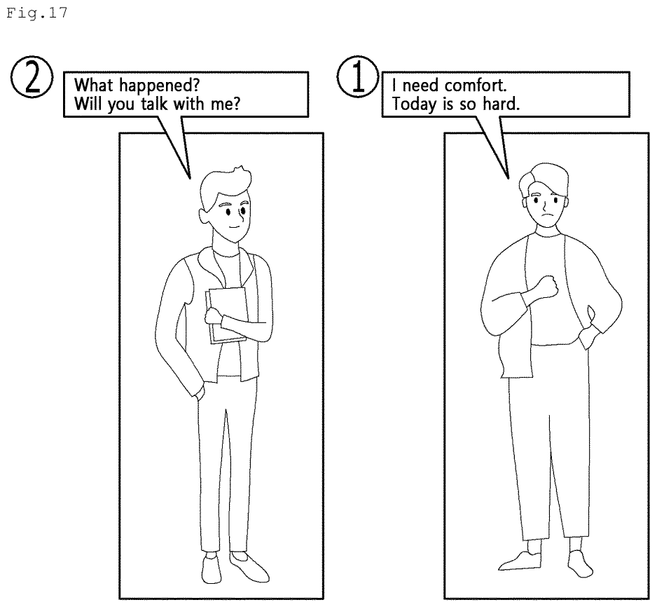

The term “digital therapeutics” used in this specification may include various types of alleviation programs that can alleviate psychological risks that the client has. For example, this may be a game-type alleviation program described with reference to FIGS. 15 and 16 or an interactive avatar alleviation program described with reference to FIG. 17. However, this is only an example and does not limit the scope of the present invention.

The artificial intelligence diagnosis unit 153 may suggest a prescription and a digital treatment based on data, which are similar to those of the counselor, but are objectively considered as being more effective, through a DB accumulated on the basis of data and opinions on specific diseases recommended by the counselor diagnosis unit 152, and analysis results of artificial intelligence.

Meanwhile, although the exemplary embodiment of the present invention is described on the basis of implementation of a method connecting a counselor selected among a plurality of counselors to a client, this is only an exemplary form. Alternatively, according to another embodiment of the present invention, psychological counseling on a client may be performed by artificial intelligence even when there is no counselor connected through the counselor terminal 300. In this case, the artificial intelligence diagnosis unit 153 may provide an artificial intelligence-recommended digital therapeutic on the basis of information acquired by the artificial intelligence in the process of conducting psychological counseling with the client, instead of information acquired by the counselor in the psychological counseling process. In this case, the first digital therapeutic may include only the artificial intelligence-recommended digital therapeutic without a counselor-recommended digital therapeutic.

The counseling center DB storage unit 154 may store predetermined psychological counseling information, such as chat logs and video logs, acquired in the psychological counseling process. The psychological counseling information may be stored as text, and in this case, it is easy to extract major keywords related to the counseling.

The call unit 155 may implement a process of retrieving predetermined information needed for counseling. For example, it may execute an operation of retrieving information on the improvement or decline type by learning date utilizing cognitive abilities, emotional abilities, and digital therapeutics.

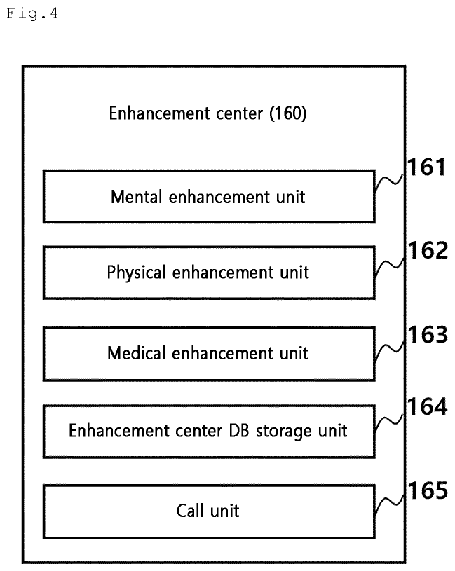

FIG. 4 is an exemplary view showing the configuration of the enhancement center 160 included in a diagnostic system providing server according to a counseling process in virtual reality according to an embodiment. As shown in FIG. 4, the enhancement center 160 may include a mental enhancement unit 161, a physical enhancement unit 162, a medical enhancement unit 163, an enhancement center DB storage unit 164, and a call unit 165.

The mental enhancement unit 161 may provide a function of comparing, analyzing, matching, and providing the client with a digital therapeutic matching an appropriate diagnosis, among the elements classified into, for example, post-traumatic stress disorder, personality disorder, sexual disorder, eating disorder, sleep disorder, adjustment disorder, impulse control disorder, and the like, as a second digital therapeutic.

The physical enhancement unit 162 may provide a function of matching and suggesting a digital therapeutic according to an appropriate diagnosis, among the elements related to physical diseases such as musculoskeletal system, dry eye, tinnitus, chronic fatigue, decreased vision, chest pain, and the like, as a second digital therapeutic.

The mental enhancement unit 161 may provide a function of matching and suggesting a digital therapeutic, which may lead to improvement of diseases such as stroke, smoking, obesity, alcoholism, migraine, cancer, and the like determined as requiring medical treatment through counseling, as a second digital therapeutic.

The enhancement center DB storage unit 164 is a repository where records of executing various types of software provided as digital therapeutics (e.g., first digital therapeutic and/or second digital therapeutic) are stored.

The call unit 165 may implement a function of transmitting a DB for the counselor or the client to confirm his or her own data from the counseling center 150 and information center 170 as needed.

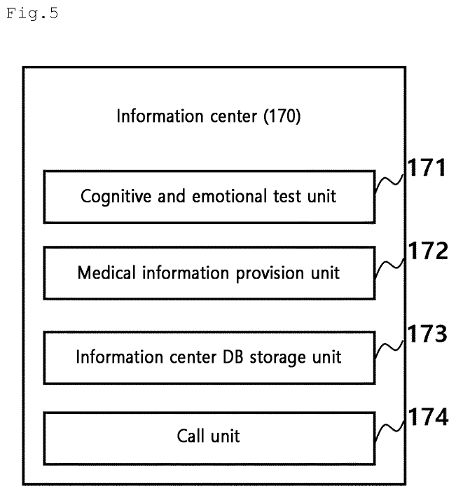

FIG. 5 is an exemplary view showing the configuration of the information center 170 included in a diagnostic system providing server according to a counseling process in virtual reality according to an embodiment. The information center 170 may also be referred to as a medical information center as it may store/record/read/retrieve medical information. As shown in FIG. 5, the information center 170 may include a cognitive and emotional test unit 171, a medical information provision unit 172, an information center DB storage unit 173, and a call unit 174.

The cognitive and emotional test unit 171 may provide a task of objectively verifying which of the cognitive and emotional abilities of the client is outstanding and insufficient through data. For example, the operation of the information center 170 allowing the counselor to perform a cognitive and emotional test may be performed on the basis of at least one among questionnaire information, behavioral information, and biometric information. For example, the questionnaire information may include information on a result of answering a predetermined questionnaire by the client, the behavioral information may include information on the response time and error rate measured by the client by executing a predetermined measurement program, and the biometric information may include information on the nonverbal elements, psychological index elements, and biometric signals of the client.

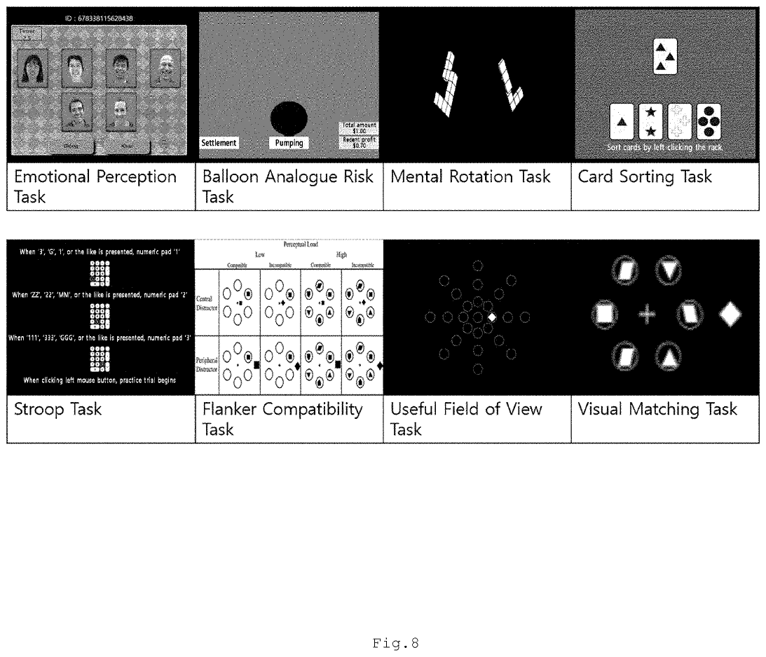

In an example, the task may be an emotion recognition task that determines different facial expressions among presented faces. For example, it can be the Emotional Perception Task shown in FIG. 8, and details thereof will be described below with reference to FIG. 8.

In an example, the task may be a behavioral inhibition and control task that earns the most profits when getting points by clicking on the intensity of a randomly inflated balloon. For example, it can be the Balloon Analogue Risk Task shown in FIG. 8, and details thereof will be described below with reference to FIG. 8.

In an example, the task may be a spatial ability task of determining whether two rotated figures are the same. For example, it may be the Mental Rotation Task shown in FIG. 8, and details thereof will be described below with reference to FIG. 8.

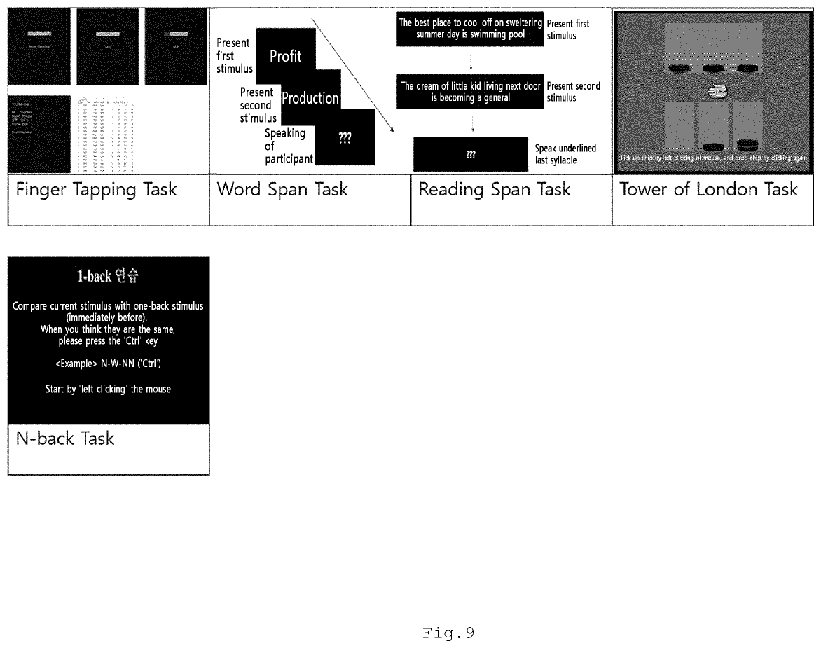

In an example, the task may be a task related to the motor ability of pressing a button most quickly during a limited time. For example, it can be the Finger Tapping Task shown in FIG. 9, and details thereof will be described below with reference to FIG. 9.

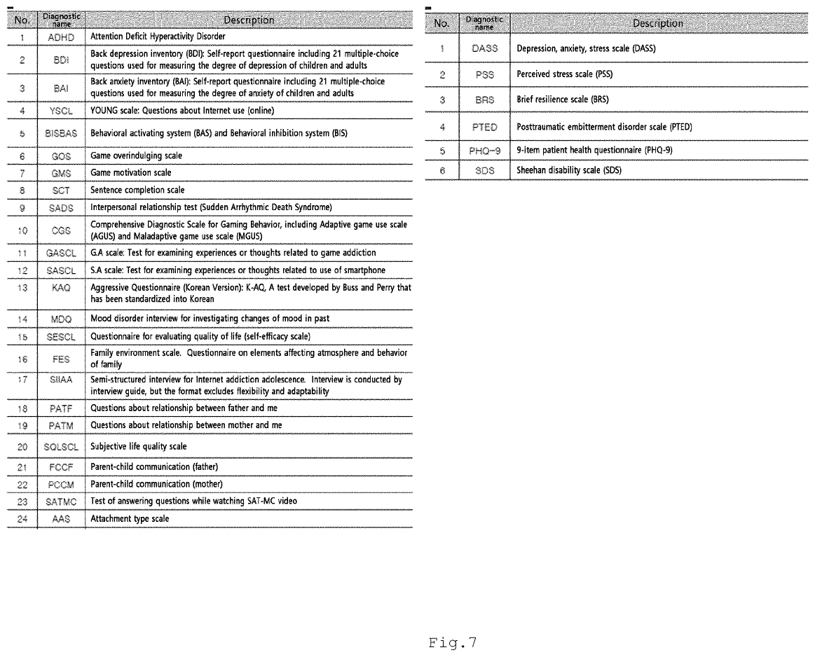

Meanwhile, the tasks described above are only exemplary implementations according to the embodiments of the present invention, and the scope of the present invention is not limited thereto. For example, implementation examples described below with reference to FIGS. 7 to 12 may be considered as other exemplary implementations of the task related to the cognitive and emotional test. This will be described in more detail in the latter part of this specification.

The medical information provision unit 172 may further provide information on the diagnosis, prescription, alternative medicine, recommended food, recommended exercise, and the like of the client determined by the counselor diagnosis unit 152 and the artificial intelligence diagnosis unit 153 of the counseling center 150.

The information center DB storage unit 173 may store medical information. The medical information stored in the information center DB storage unit 173 may include raw data related to the cognitive and emotional test of the client, and analysis result data thereof. In addition, the medical information stored in the information center DB storage unit 173 may further include academic information updated through medical seminars.

For example, the server 100 for constructing the system according to an embodiment of the present invention may provide the client with the counseling result according to latest medical techniques on the basis of academic information such as medical seminars or the like held by experts including psychiatrists.

The academic information may be updated whenever medical seminars are held or may be updated at predetermined intervals (e. g., quarterly or monthly). The academic information may be individually updated to be independent from the operations performed in the counseling center 150 and the enhancement center 160.

Meanwhile, when the academic information stored in the information center DB storage unit 173 is updated, the customized information of each client may be collectively changed on the basis of the updated academic information. As the customized information of each client is collectively updated in accordance with the time point of updating the academic information, the customized medical information of each client may be managed as data based on the latest academic information at all times.

The information center DB storage unit 173 may further include information related to precautions needed for supporting the client, and this may be included in customized information of each client. The information related to precautions may be information provided to the guardian of the client or information provided directly to the client.

Meanwhile, the medical information stored in the information center DB storage unit 173 may be updated at regular intervals, or may be individually updated, when there is a person who views the DB, at the time of viewing or immediately after viewing health information corresponding to the person.

The call unit 174 may call various medical information stored in the information center DB storage unit 173 in response to a specific request (e.g., a request from the counseling center 150). Alternatively or in parallel thereto, the call unit 174 may perform a function of providing medical information contrasted with medical keywords as customized information on the basis of search based on medical keywords appeared in the psychological counseling process.

FIG. 6 is a flowchart illustrating the steps of the operation performed by a diagnostic system providing server according to a counseling process in virtual reality according to an embodiment. Although the steps shown in FIG. 6 may be performed in the order indicated by the arrows, this is not limited thereto, and it should be understood that at least some of the steps may be performed in parallel with other steps or may be performed independently.

Step 601 is the step where a client connects to the server 100, which implements a counseling platform, through the client terminal 200, such as a PC, a mobile device, or the like. When the client connects to the server 100 through the client terminal 200, at step 602, the server 100 may allow the client to input basic information related to the client, such as the gender, height, weight, and the like. The basic information may include questionnaire information, behavioral information, and biometric information of the client.

Although the server 100 may also allow the client to directly input basic information at step 602, this is only an example. When the client has a history of conducting a series of psychological counseling and conducting digital therapeutics in advance according to an embodiment of the present invention, previously stored basic information may be reused as basic information.

At step 603, the server 100 may identify whether the client has a history of performing a cognitive and emotional task. When the server 100 stores a log of the task previously performed by the client (e.g., when related log information is confirmed from the counseling center DB storage unit 154, the enhancement center DB storage unit 164, the information center DB storage unit 173, etc.), the appointment and counseling conducted in the counseling center appointment and generation unit 151 of the counseling center 150 may be proceeded. When there is no task log, the cognitive and emotional test of step 606, which is one of the sub-steps of step 605 performed by the information center 170, may be performed.

For example, the cognitive and emotional test unit 171 of the information center 170 may allow the client to perform a cognitive and emotional test. The cognitive and emotional test may be an operation of the information center that perform psychological risk assessment on the client information using a psychological assessment tool previously stored in the server 100. For example, the psychological risk assessment may derive information on psychological illness of the client, information on a psychological treatment method suitable for the client, or the like. In addition, for example, the previously stored psychological assessment tool may include a survey tool that perform psychological risk assessment by deriving a survey result from the questionnaire information of the client, a measurement program that performs psychological risk assessment on the client on the basis of the behavioral information of the client, and various artificial intelligence neural network models that perform psychological risk assessment on the basis of the biometric information of the client. In relation thereto, detailed descriptions of the survey tool, the measurement program, and the neural network model will be described in more detail with reference to FIGS. 7 to 12 in the latter part of this specification.

Data related to the client such as the response time and error rate can be acquired through the task performed by the cognitive and emotional test unit 171, and elements affecting the counseling may be extracted through the data. The extracted elements may be transmitted to the counseling center 160 by the information center 170, and this may lead to the process of making an appointment and conducting counseling (e.g., step 604) of the counseling center 160 after the information provision process (e.g., step 605) of the information center 170.

At step 604, the counseling appointment can be made by allowing the client to input information on the counseling topic, counseling method (e.g., chat counseling, avatar counseling, video counseling, etc.), counseling date and time, counseling session, counseling duration, counselor, and the like desired by the client. Through the appointment, a counseling room can be created in a virtual space.

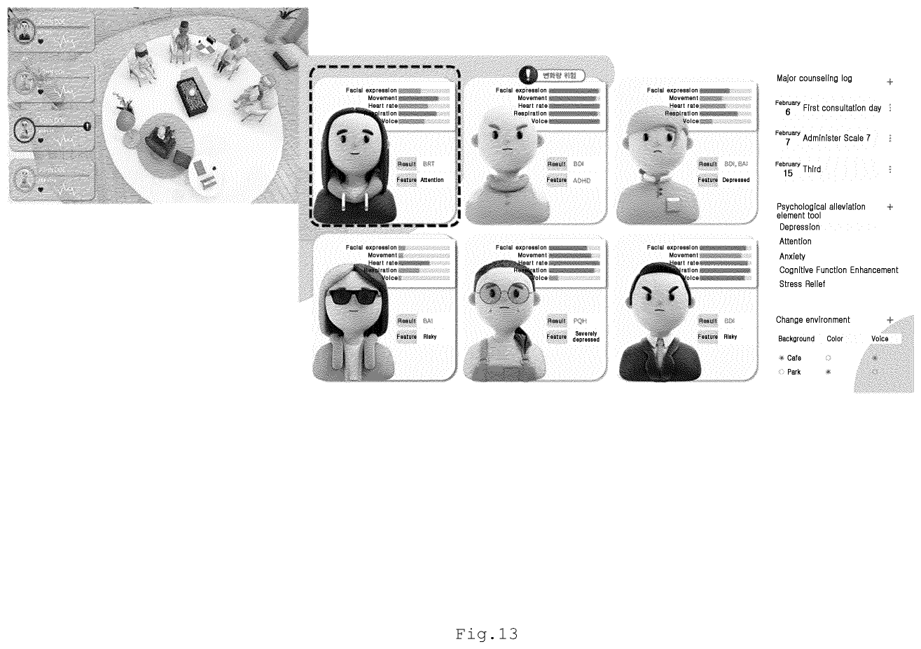

In the counseling room created in a virtual space (e.g., a counseling room in the metaverse), counseling may be conducted using non-face-to-face avatars or avatar chat. An example of a metaspace provided by the server according to an embodiment of the present invention to connect a client and a counselor is shown in FIG. 13. Details thereof will be described below in the latter part of this specification with reference to FIG. 13.

When counseling is provided in a virtual space, various technical effects can be obtained. For example, according to a result of research, it is understood that when an exposure therapy for generalized social anxiety disorder is performed in virtual reality, 93% of participants show high satisfaction with the treatment and therapist in the virtual environment as the results of online self-report tests and telephone interviews performed before the experiment, during the experiment, after the experiment, and 3 months after the experiment, and social anxiety disorder symptoms are reduced after the treatment compared to before the treatment.

In addition, there is a result of research showing that when a client receives counseling using a VR avatar in virtual reality, the client forms rapport more quickly, feels emotionally comfortable, and is empathetically understood compared to face-to-face counseling. Furthermore, there is also a result of research showing that as a result of conducting counseling twice a week, a total of four sessions, in a way of discussing specific topics with the VR avatar in virtual reality, anxiety about the fearful situation of conversation with opposite sex has decreased, positive automatic thoughts have increased, and heart rates before conversation and the rating score of the observer about the performance anxiety have been decreased significantly compared with a waitlist control group. Furthermore, there is a result of survey showing that when a digital human is utilized as a virtual counselor, SimSensei Kiosk utilizing the digital human has a positive effect on the digital human to break down psychological barriers and share its information.

At step 604, a log related to the process of conducting counseling in a virtual reality space (e.g., a virtual counseling room) that may provide unique technical effects as described above may be stored in the counseling center DB storage unit 154.

When the counseling is completed, an operation of analyzing and recommending a counseling result is performed at step 609. At step 610, the server 100 may receive a cognitive and emotional test result of the client from the counselor, as well as the definition, prescription, and digital therapeutics for the disease subjectively determined and recommended by the counselor. This may be referred to as a counselor-recommended digital therapeutic in this specification. The operation of step 610 may be performed by the counselor diagnosis unit 152. Alternatively or in parallel thereto, at step 611, the server 100 may receive the definition, prescription, and digital therapeutics for the disease determined and recommended by artificial intelligence. The artificial intelligence may collect a database of determinations made by various counseling experts from the counseling center DB storage unit 154, and recommend only the prescriptions and digital therapeutics of the artificial intelligence. This may be referred to as an artificial intelligence-recommended digital therapeutic in this specification. According to an embodiment of the present invention, the counselor-recommended digital therapeutic and the artificial intelligence-recommended digital therapeutic may be provided to the client together.

The client may re-execute the information provision step (e.g., step 605) of the information center on the basis of the result of the psychological counseling and result of the digital therapeutic recommendation. In this case, a cognitive/emotional test step (e.g., step 606) corresponding to a sub-step of step 605 may be re-executed. Alternatively, a customized information provision step (e.g., step 607) or an academic information provision step (e.g., step 608) corresponding to a sub-step of step 605 may be performed. Predetermined medical information provided through these information provision steps may be transmitted to the enhancement center 160 and affect subsequent steps (e.g., step 612) performed by the enhancement center 160.

Meanwhile, the cognitive/emotional test process of step 606 may be a cognitive/emotional test process that the client performs as described above.

The customized information provision process of step 607 may be a process of obtaining information by retrieving customized information about the client from the storage center DB storage unit 173 on the basis of the result of the psychological counseling and at least one of the first digital therapeutics recommended by the counseling center.

The academic information provision process of step 608 may be a process of providing information on the latest medical techniques provided on the basis of academic information such as medical seminars held by various experts including psychiatrists, psychological counselors, and the like.

At step 612, the enhancement center 160 interacts with the information center 170 to feed and forward the input and output therebetween. The enhancement center 160 may execute only the first digital therapeutic (step 613), but it may also allow the client to select and execute the second digital therapeutic (step 614). The second digital therapeutic (e.g., other digital therapeutics) may be selected from a digital therapeutic candidate group the same as that of the first digital therapeutic (e.g., recommended digital therapeutic).

The enhancement center 160 may feed and forward by recording a result of performing at least one among the first digital therapeutic (e.g., recommended digital therapeutic) and the second digital therapeutic (e.g., other digital therapeutics), and changing and storing customized information stored in the information center DB storage unit 173 of the information center 170 on the basis of the recorded result of performing the digital therapeutic.

In the enhancement center 160, the client may improve cognitive and emotional abilities in various fields by performing recommended digital therapeutics (e. g., step 613) and/or performing client-selected digital therapeutics (e.g., step 614) on the basis of the acquired counseling results, recommendations, and medical information of the information center.

The digital therapeutics may include a game-type alleviation program provided through the embodiment according to FIGS. 15 and 16. Alternatively, the digital therapeutics may include an interactive avatar alleviation program provided through the embodiment according to FIG. 17. Although the digital therapeutics provided through the embodiments of the present invention may include various types of alleviation programs that may alleviate the psychological risk of the client, this is only an example and does not limit the scope of the present invention. The descriptions of FIGS. 15 to 17 will be described below in more detail.

Log records related to the digital therapeutics (or alleviation programs included in the digital therapeutics) executed in the enhancement center 160 may be used to provide and change various personalized medical information in the information center 170. As described above, as is affected by the feed and forward process according to the interaction between the enhancement center 160 and the information center 170, information according to more closely related personal medical information may be provided.

When information acquisition and task performance in the enhancement center 160 and information center 170 are completed, the client is allowed to make a follow-up counseling appointment at step 615 according to a previously planned session, and the client is allowed to visit again at a later time.

The steps described with reference to FIG. 6 are only a preferred embodiment for achieving the objects of the present invention, and some steps may be added or deleted as needed, and any one step may be included and performed in another step. The order of each operation disclosed in FIG. 6 is only an order arranged for convenience of understanding, and this order is not limited to a time-series order, and the operation may be performed in a different order changed according to selection of the designer.

FIG. 7 is an exemplary view showing a survey tool for deriving a survey result based on questionnaire information of a client according to an embodiment.

Referring to FIG. 7, the server 100 may previously store the survey tool of FIG. 7, which can identify psychological risk factors of a client. The server 100 may derive a previously stored psychological risk assessment using the previously stored survey tool at step S1010 in response to the result of the questionnaire of the client. The psychological risk assessment may include information on the psychological illness of the client, information on a psychological treatment method suitable for the client, or the like.

FIGS. 8 and 9 are exemplary views showing a measurement program that perform psychological risk assessment of a client on the basis of behavioral information of the client according to an embodiment.

Referring to FIGS. 8 and 9, the server 100 may store a measurement program capable of identifying psychological risk factors of a client, and the behavioral information may include information on the process or result of the measurement program provided by the server 100 and performed by the client.

The measurement program according to the example of FIG. 8 is as described below.

The ‘Emotional Perception Task’ shown in FIG. 8 is a task of confirming the ability of recognizing and processing emotions. A client confirms whether all the faces of the people shown on the screen have the same facial expressions, and presses the “same” button when all the facial expressions are the same, and presses the “different” button when the facial expressions are different. This measurement program shows a rapid response to a stimulus emotionally consistent with the state of the client. In addition, it is reported that as those who suffer from a depressive disorder or an emotional disorder tend to have a difficulty in accurately recognizing facial expressions, the response time is relatively long, and the accuracy is low. (References: Dawson et al., 2002; Kring & Campellone, 2012).

The ‘Balloon Analogue Risk Task’ shown in FIG. 8 is a task of measuring the tendency of the impulse and control ability of a client. Although there is a risk of bursting, it is a task of earning a profit in proportion to the inflation size of the balloon, and the average number of clicks succeeded in accumulating coins according to the balloon color is recorded in each round of trial. When a pumping button is pressed, yellow, orange, and blue balloons gradually inflate according to the number of clicks. When the client presses a payout button, an amount as much as inflating the balloons is accumulated in proportion to the number of times of pressing the button. However, the balloons have a different maximum strength according to the color, and when it is attempted to inflate a balloon more than the maximum strength, the balloon bursts, and the profit for the balloon may not be accumulated. In addition, the balloons may burst at random time points. (see: Lejuez, Read, et al.; Kahler, 2002).

The ‘Mental Rotation Task’ shown in FIG. 8 is a task of understanding the internal perceptual process that occurs when an object is presented at a different angle by rotating the shape. Seeing two three-dimensional figures shown on the screen, the participant should mentally determine whether the two figures are the same. There are six rotation angles of 0, 30, 60, 90, 120, and 180 degrees, and when the rotation angle is closer to 90 degrees, it is more difficult to determine. A result of research is reported that the better the spatial ability of the right brain, the derived answer is faster and more accurate. (see: Johnson, 1990; Pietsch, & Jansen, 2012).

The ‘Card Sorting Task’ shown in FIG. 8 is a task of identifying the execution function that the frontal lobe has. It is a task of sorting cards by inferring through trial and error without being informed of the rules of the presented cards. This measurement program may confirm how well the cognitive control is performed on the frontal lobe by measuring the delay time with respect to the “perseveration phenomenon” that insists on the current response or determination. (Reference: Fox, Mueller, Gray, Raber, & Piper, 2013).

The ‘Stroop Task’ shown in FIG. 8 is a task presented to test cognitive control abilities, such as the execution function, processing speed, memory capacity, and the like. When a single-digit appears, input 1, when a two-digit number appears, input 2, and when a three-digit number appears, input 3. However, when a different number that induces conflict with a specific-digit number is presented, automatic processing should be suppressed. (References: Lamers et al., 2010; van der Sluis et al., 2004).

The ‘Flanker Compatibility Task’ shown in FIG. 8 is a task of measuring the level of visual attention. When the eyes are focused after a cross shape appears at the center of the screen, figures are presented around the center. Six circles and several figures rapidly appear and disappear around the center, and the figures are included or not included in these circles. The client quickly identifies the circles presented for a short time and determines whether a square or a diamond is within the circles. The square or diamond may also be outside the circles, and these figures should be ignored. When figures other than the square or diamond are within the circles, since the difficulty increases as the number of figures increases and the difficulty decreases as the number of figures decreases, and the higher the attention increases, the number of visible objects increases, it is difficult to find the target stimulus, and the correct answer rate is lowered (see Miller & Jeff, 1991; Green, Shawn et al., 2003).

The ‘Useful Field of View Task’ shown in FIG. 8 is a task of finding out the extent of the visually perceptible area that can be recognized at a glance without movement of the eyes or head. When a diamond-shaped stimulus appears and disappears in the middle of 24 points according to the angles and distances in 8 directions presented with respect to the center, it is required to answer the direction in which the stimulus has appeared through the number pad on the keyboard. When a mask stimulus that eliminates afterimages appears and disappears after the stimulus on the eye disappears, the client receives a response. This implies that the better the client perceives the direction of stimulus and the farther the stimulus appears from the center, the broader the sensitivity of the visual perception. (Reference: Sekuler, Bennett, & Mamelak, 2000).

The ‘Visual Matching Task’ shown in FIG. 8 is a task of confirming the visual attention and control ability. It is a task of presenting a picture for a short time while the client is focusing on the screen, and determining whether the figures in the six circles are the same as or different from the shape on the left or right outside the circles. (Kim Sang-yeop, Jeong Jae-beom et al., 2017).

The ‘Finger Tapping Task’ shown in FIG. 9 is a task of repeatedly tapping a button as quickly and accurately as possible with the index finger of either the left or right hand within the given time. Through the task, the motor function and motor control ability of the client can be identified. For example, since a patient with Parkinson's disease does not smoothly perform the motor function, the patient cannot repeatedly but intermittently tap the key, or may not stop moving although the time limit has passed. (Duchek, Balota, & Ferraro, 1994).

The ‘Word Span Task’ shown in FIG. 9 is a task of measuring the working memory ability of memorizing language-related words. This is a task of reading and memorizing, when a question mark appears after words are presented sequentially at the center of the screen, the presented words, and sequentially speaking the words aloud. Whether the client speaks the words in a correct pronunciation in a correct order is assessed. (see: La Pointe & Engle, 1990; Caplan, Rochon, & Waters, 1992)

The ‘Reading Span Task’ shown in FIG. 9 is a task of measuring the working memory ability of memorizing language-related phrases. Sentences are presented several times at the center of the screen. The client is asked to read the presented sentence aloud and memorize the last phrase. When a question mark appears, the client is asked to speak the last words of the presented sentence in order. Whether the client speaks the last words correctly in order is assessed. (see: Friedman, & Miyake, 2004; Daneman, & Hannon, 2007).

The ‘Tower of London Task’ shown in FIG. 9 is a task of measuring the metacognitive ability. This is a task in which the client sees the blocks stacked in a predetermined pattern in the upper part of the screen and move the blocks in the lower part in the same shape as quickly as possible. The greater the cognitive control, the shorter the delay time of moving the blocks, and the shorter the time required to complete the task. (see Shallice, 1982; Boot, Kramer, Simons, Fabiani, & Gratton, 2008).

The ‘N-back Task’ shown in FIG. 9 is a task of identifying working memory and fluid intelligence. It is a task in which the client memorizes letters presented on the monitor together with speech sound, recalls the stimulus presented N-time before (e.g., when presented in order of g->z->t->z, 1-back=t, 2-back=z, and 3-back=g), and responds by pressing the keyboard when the presented letters are the same. The client determines whether the letter currently presented on the screen is the same as the letter presented N-time (1, 2, 3) before, and responds using the keyboard. As the load allocated to the working memory increases with increase of N, the difficulty increases. (Reference: Jaeggi, Buschkuehl, Jonides, & Perrig, 2008).

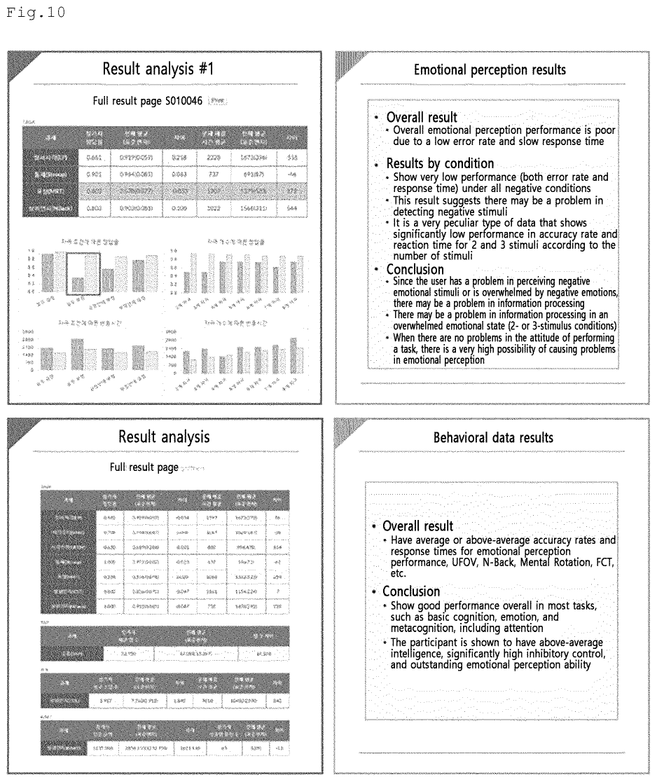

FIG. 10 is an exemplary view showing a result of performing a previously stored psychological risk assessment in response to a result of executing a measurement program according to an embodiment.

Referring to FIG. 10, on the basis of information on the response time and error rate measured by the client by executing the measurement program, the server 100 may output a result of the measurement based on preset scoring criteria according to the measured response time and error rate.

Meanwhile, biometric information of the client may include nonverbal elements, psychological index elements, and biometric signals. An embodiment of performing psychological risk assessment by the server 100 on the basis of the nonverbal elements, psychological index elements, and biometric signals of the client is described below.

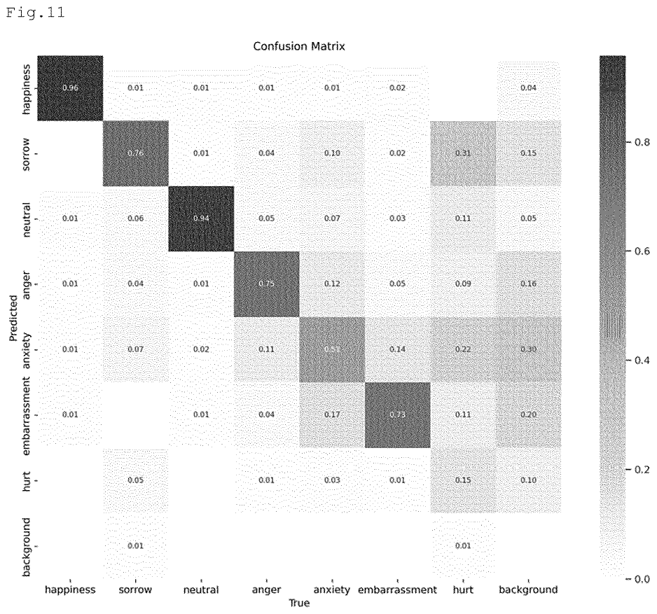

FIG. 11 is an exemplary view showing a neural network model trained to perform psychological risk assessment of a client on the basis of nonverbal elements (e.g., facial expressions) of the client according to an embodiment.

Referring to FIG. 11, the server 100 may acquire a real-time image of the client from the client terminal 200, and the server 100 may determine the psychology of the client on the basis of a neural network model (e.g., YOLO) trained to classify the psychology of the client through the acquired image. Furthermore, the server 100 may improve the accuracy and speed by utilizing the Swin Transformer in the areas where accuracy of the YOLO model is required.

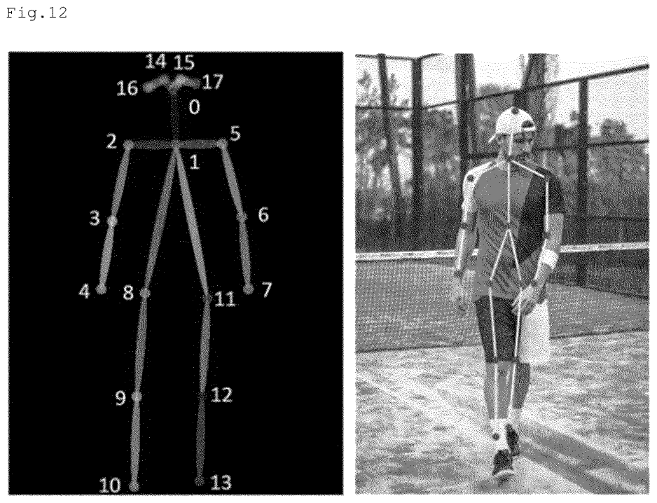

FIG. 12 is an exemplary view showing a neural network model trained to perform psychological risk assessment on the basis of psychological index elements (e.g., body movement) of a client according to an embodiment.

Referring to FIG. 12, the server 100 may acquire a real-time image of the client from the client terminal 200, and the server 100 may extract feature points of the client's body based a movement determination algorithm (e.g., Alphapose), and determine the psychology of the client according to the nonverbal elements of the client through a neural network model based on a Convolution Neural Network (CNN) trained to determine the motion of the client on the basis of the movement of the feature points. For example, the neural network model may be proceeded in a way of finding feature points of a person defined in advance in a method of finding the postures of a person or animal in an image, and after finding the feature points, it may finally detect whether there is a specific posture by identifying the relationship between the feature points, such as the position and angle. At this point, the feature points may be set as feature points of the face, as well as simple joints, and may be further subdivided according to the interest of the designer.

In addition, the server 100 may determine the psychology of the client on the basis of the biometric signal (e.g., voice) of the client. For example, the server 100 may acquire speech sound of the client, and determine the psychology of the client according to the psychological index elements (e.g., voice) of the client through a neural network model based on a Convolution Neural Network (CNN) trained to determine the psychology of the client through the acquired speech sound.

As described above, at step 604 of FIG. 6, the server 100 may provide a virtual reality space (e.g., metaverse space) connecting a client and a counselor, and perform a counseling center operation of determining an alleviation program for alleviating the psychological risk of the client.

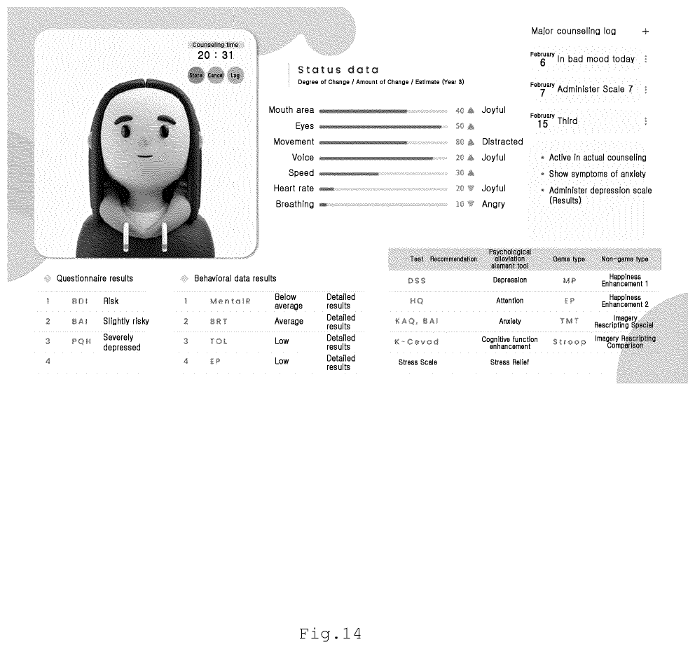

FIG. 13 is an exemplary view showing a metaspace provided by a server 100 to connect a client and a counselor according to an embodiment, and FIG. 14 is an exemplary view showing a client profile according to an embodiment.

Referring to FIGS. 13 and 14, the server 100 may provide a metaverse space that connects a plurality of clients having common psychological risk factors with a counselor who will conduct counseling on the basis of the psychological risk assessment of the client. In addition, the server 100 may provide the counselor with a profile including information on the client and psychological risk assessment information. The server 100 may provide the client with an alleviation program determined by the counselor through counseling between the client and the counselor. In addition, the server 100 may provide the client with a previously matching alleviation program based on the result of the psychological risk assessment.

As described above, at steps 610 and 611 of FIG. 6, the counseling center 150 may recommend at least one first digital therapeutic (e.g., counselor-recommended digital therapeutic or artificial intelligence-recommended digital therapeutic) including an alleviation program, and at steps 613 and 614, the enhancement center 160 may allow the client to perform the first digital therapeutic (e.g., counselor-recommended digital therapeutic or artificial intelligence-recommended digital therapeutic (collectively, recommended digital therapeutic)) including an alleviation program and a second digital therapeutic (e. g., another digital therapeutic) including another alleviation program. For example, the server 100 may provide the alleviation programs of FIGS. 15 and 16 in the metaspace.

FIGS. 15 and 16 are exemplary view showing a game-type alleviation program that helps alleviate psychological risk factors of a client according to an embodiment.

The ‘Eye-hand coordination: belly of the beast’ shown in FIG. 15 is a task of confirming the psychomotor mechanism, and may confirm spatial perception and motor function by estimating the direction of moving and avoiding obstacles.

The ‘Working memory: Electric Shock’ shown in FIG. 15 is a task of observing electric sparks charged and discharged in four generators as many as a predetermined number of times on the screen, and finally selecting a generator where the most electric sparks are charged, and since the number of sparks charged and discharged in the generator should be accurately memorized, it is possible to measure and improve the working memory, which is a type of short-term memory related to integration, processing, deletion, and reproduction of temporary information.

The ‘Divided attention: faster food’ shown in FIG. 15 is a task of reconstructing a dual task, which measures the divided attention ability of allocating attention resources into multiple tasks and managing the resources, into a task form. The dual task uses a monitoring task and a task that uses the ability of visual search and spatial construction. The divided attention can be measured on the basis of the performance level of each task when the task is performed individually rather than as a dual task and how much the performance is lowered when the dual task is performed.

The ‘High cognition, rule shift: friend or foe’ shown in FIG. 15 is a task in which the client becomes a blue monster on the human side holding a ray gun or a key remote controller or a white monster on the alien side, and observes human aliens appearing in four places on the screen, subdues the enemy side having a gun using the ray gun, and rescues tied up allies using the key remote controller. Since the client's side, equipment, and targets appearing in the four places change randomly and thus the client should quickly grasp the changing rules and take appropriate responses, it is possible to measure and improve the cognitive ability responding to the change of the rules.

The ‘Spatial recognition, configuration: image match’ shown in FIG. 15, in which spatial memory is memory related to a space as the name implies, measures the ability of memorizing a place where something is located or something that is located at a place. This program is a task of presenting pairs of identical pictures in a 4×4 grid, a total of 16 pictures, on the screen, and making the client memorize locations of the pictures well and select the same pictures in pairs after the pictures are hidden for a moment. It is possible to measure and improve the spatial memory by testing whether the client can remember the locations of pictures well and training the client accordingly.

The ‘Visual Attention: Impatient Shopper (ADHD treatment and alleviation tool)’ shown in FIG. 15 is a task of enhancing visual attention, and description of a target stimulus is presented in simple words for variation with a measurement task or the like. The target is presented in plurality, from 1 to 4, and the user should find a corresponding stimuli as quickly as possible. Stimuli are randomly arranged within four categories such as color, shape, and the like.

The ‘Word recognition: Magician Tower’ shown in FIG. 15 is a psychological process of recognizing visually presented words, i.e., word recognition, and grasping their meanings. This program is a task of remembering words presented on a scroll and finding the words presented on the scroll among the words engraved on the wall that appears when the scroll disappears, and it measures the ability of recognizing, not remembering, words.

The ‘Emotion perception: smile for the camera’ shown in FIG. 15 is a task designed to enhance emotion perception ability, and it is a task of showing faces of several people to a user at once and determining by the user whether all the presented facial expressions of the people are emotionally positive or negative. For thrills and fun, a time limit is presented in each trial, and the number of pictures is adjusted to take into account the difficulty level.

The ‘Selective attention, TMT: Stadium Slugger’ shown in FIG. 16 is a task of mainly measuring the selective attention ability of a user on the basis of a Trail Making Test (TMT). The goal is to enhance persistency by increasing the fun as a task by applying the rules of the classic TMT. It can be applied to measuring visual attention, memory, and like.

The ‘Finger tapping: Stop UFO Showdown’ shown in FIG. 16 is a task of protecting a player from falling off the cliff and bouncing off a rocket-shaped UFO that pushes the player toward the cliff, by tapping the UFO to a predetermined beat. The motor control ability function impaired by emotional function-related diseases, such as MDD, schizophrenia, Parkinson's disease, and the like, can be measured by confirming whether the player may respond at a set timing and beat, and based on this, risk groups with the possibility of emotional function-related diseases can be screened, and the emotional function can be improved through repeated play.

The ‘Short-term memory, digit span: ticking time bomb’ shown in FIG. 16 is a task of which the goal is to memorize well the digits of bomb-disarming password listed clockwise around a bomb and input the password in an order reverse to the listed order so that the bomb does not explode. The number of digits to memorize increases as the task succeeds, and when the task fails, the number of digits decreases. Accordingly, it is possible to measure and enhance the capacity of working memory, and as the difference between the people with low ability and high ability is clear, it is further easier to measure.

The ‘Stroop test: true or lie’ shown in FIG. 16 is a task of measuring and improving the frontal lobe inhibitory ability.

The ‘Emotion perception: ancient robot’ shown in FIG. 16 is a task designed to measure the emotional recognition ability. Only one stroke of stimulus is used to speed up the process, and the emotional recognition ability is measured mainly through the response time and correct response ratio of the user. Since the task itself is very simple, it is designed to allow repetitive training by increasing persistency as a task.

FIG. 17 is an exemplary view showing an interactive avatar alleviation program that helps alleviate psychological risk factors of a client according to an embodiment.

Referring to FIG. 17, the server 100 may provide an enhancement program that provides empathetic conversation between a client avatar and another artificial intelligence avatar based on an artificial intelligence model.

According to FIG. 17, the server 100 may create an avatar having an appearance and a voice selected by the client. The server 100 may acquire speech sound of the client and control the client avatar to speak the speech sound of the client in the voice selected by the client. In addition, the server 100 may create an artificial intelligence avatar separate from the avatar controlled by the client. For example, the artificial intelligence avatar can be created based on a large vision language model (e. g., hereinafter, referred to as an “AI model”) that responds to the speech sound of the client. In addition, the appearance of the artificial intelligence avatar can be generated by applying a digital human image. A digital human is a technique of creating the appearance of the AI model so that the AI model that outputs text, images, audio, video, and the like may have a three-dimensional image in the form of a human, and may be created to have predetermined gestures, facial expressions, gazes, and speech sounds according to the text output by the AI model. The server 100 inputs the speech sound of the client into the AI model so that the AI model may generate a response thereto, and then controls the artificial intelligence avatar so that the artificial intelligence avatar having a digital human image may output the response and make an empathetic conversation with the client.

As an additional embodiment, in FIG. 17, the server 100 may create an artificial intelligence avatar having a preset appearance and voice that matches the appearance and voice of the client avatar, on the basis of information on the appearance and voice of the avatar selected by the client as his or her avatar. For example, when the client avatar has the appearance of a 20-year-old male, the artificial intelligence avatar may be set to have the appearance of a 20-year-old female.

As an additional embodiment, in FIG. 17, the server 100 may acquire biometric information of the client responding to the answer of the artificial intelligence avatar, and modify the appearance and voice of the artificial intelligence avatar. For example, in this process, the server 100 may acquire nonverbal elements, psychological index elements, and biometric signals of the client in the manner described with reference to FIGS. 11 and 12, and may determine whether the client's response to the biometric information is a positive response (e.g., expression of happiness, expression of liking, laughter, etc.) or a negative response (e.g., frowning, avoiding eye contact, etc.) by utilizing the psychological assessment tool described above. Accordingly, the server 100 may determine a first appearance and a first voice, in which the biometric information of the client in response to the answer of the artificial intelligence avatar is determined as a positive response, while using a plurality of various appearances and voices during the process of executing an alleviation program. Accordingly, the server 100 may re-create the appearance and voice of the artificial intelligence avatar by applying the first appearance and first voice to the artificial intelligence avatar to output an answer to the speech sound of the client. At this point, the appearance of the artificial intelligence avatar can be created by combining a plurality of genders, a plurality of ages, and a plurality of appearances.

As an additional embodiment, in FIG. 17, the server 100 may control the artificial intelligence avatar to take determined gestures, facial expressions, and gazes on the basis of the gestures, facial expressions, and gazes determined by the agent of a reinforcement learning model while the artificial intelligence avatar outputs an answer. For example, the server 100 may select gestures, facial expressions, and gazes that the artificial intelligence avatar should take on the basis of the reinforcement learning model while the artificial intelligence avatar outputs an answer of the large vision language model, and control the gestures, facial expressions, and gazes of the artificial intelligence avatar.

The reinforcement learning model of the embodiment described above can be designed based on the Deep-Q-Network algorithm. In this case, the reinforcement learning model may include a policy function for determining the criteria for selecting, by the agent of reinforcement learning, the gestures, facial expressions, and gazes that the artificial intelligence avatar should take on the basis of the content of the answer determined by the AI model. The reinforcement learning model may include a value function that is set to receive a reward proportional to the positive biometric signal of the client according to the gestures, facial expressions, and gazes determined by the agent. For example, the value function may be set to receive a higher reward as the number of times of determining the positive biometric information of the client (e.g., expressions of happiness, affection, laughter, etc.) increases according to the gestures, facial expressions, and gazes determined to output an answer. The agent of reinforcement learning may update the criteria (e.g., parameters) for selecting the policy function to be improved in the direction of increasing the reward of the value function. Accordingly, the device 100 may control the gestures, facial expressions, and gazes that the artificial intelligence avatar should take on the basis of the reinforcement learning model based on the reward of the positive biometric signals of the client, and therefore, the psychological risk factors of the client can be efficiently improved by increasing concentration of the user on the alleviation program.

According to the embodiments described above, the present invention may allow a client and a counselor to conduct psychological service in a non-contact manner by utilizing a metaverse space, and provide a comprehensive psychological treatment platform construction technique, from the process of identifying psychological risk factors of the client on the basis of questionnaire/behavioral/biometric information related to the client, to the enhancement process of alleviating the psychological risk factors of the client by conducting counseling.

It should be understood that various embodiments of this document and the terms used herein are not intended to limit the technical features described in this document to specific embodiments, but include various modifications, equivalents, or substitutes of the embodiments. In connection with the description of drawings, similar reference numerals may be used for similar or related components. The singular form of a noun corresponding to an item may include one or more items, unless the related context clearly indicates otherwise.

In this document, each of phrases such as “A or B”, “at least one among A and B”, “at least either A or B”, “A, B, or C”, “at least one among A, B, and C”, and “at least either A, B, or C” may include all possible combinations of the items listed together in a corresponding phrase among the phrases. Terms such as “1st”, “2nd”, “first”, or “second” may be used only to distinguish a corresponding component from another corresponding component, and do not limit the components in any other aspect (e.g., importance or order). When a predetermined (e.g., a first) component is referred to as being “coupled” or “connected” to another (e. g., a second) component with or without a term such as “functionally” or “communicatively”, it means that the component may be connected to another component directly (e.g., wired), wirelessly, or through a third component.

The term “module” used in this document may include a unit implemented in hardware, software, or firmware, and may be used interchangeably with terms such as logic, logic block, part, or circuit. A module may be an integrally configured component, or a minimum unit of a component or a portion thereof that performs one or more functions. For example, according to an embodiment, a module may be implemented in the form of an application-specific integrated circuit (ASIC).

Various embodiments of this document may be implemented as software (e.g., a program) including one or more commands stored in a storage medium (e.g., a memory) that can be read by a device (e.g., an electronic device). The storage medium may include a random-access memory (RAM), a memory buffer, a hard drive, a database, an erasable programmable read-only memory (EPROM), an electrically erasable read-only memory (EEPROM), a read-only memory (ROM), and/or the like.

In addition, the processor in the embodiments of this document may call at least one command among one or more stored commands from the storage medium and execute the command. This allows the device to operate to perform at least one function according to the called at least one command. The one or more commands may include a code generated by a compiler or a code that can be executed by an interpreter. The processor may be a general-purpose processor, a Field Programmable Gate Array (FPGA), an Application Specific Integrated Circuit (ASIC), a Digital Signal Processor (DSP), and/or the like.

The storage medium that can be read by a device may be provided in the form of a non-transitory storage medium. Here, ‘non-transitory’ only means that the storage medium is a tangible device and does not include signals (e.g., electromagnetic waves), and this term does not distinguish the cases where data is stored semi-permanently on the storage medium from the cases where data is stored temporarily.

The method according to various embodiments disclosed in this document may be provided to be included in a computer program product. The computer program product may be traded between a seller and a buyer as goods. The computer program product may be distributed in the form of a machine-readable storage medium (e.g., a compact disc read only memory (CD-ROM) ), or may be distributed online (e.g., downloaded or uploaded) through an application store (e.g., Play Store) or directly distributed between two user devices (e.g., smartphones). In the case of online distribution, at least a part of the computer program product may be at least temporarily stored in a machine-readable storage medium, such as a memory of a manufacturer's server, an application store's server, or a server, or may be temporarily generated.

According to various embodiments, each component (e.g., a module or a program) of the components described above may include a single or a plurality of entities. According to various embodiments, one or more of the components or operations of the components described above may be omitted, or one or more other components or operations may be added. Alternatively or additionally, a plurality of components (e.g., modules or a programs) may be integrated into a single component. In this case, the integrated component may perform one or more functions of each of the plurality of components in a way identical or similar to those performed by the corresponding component among the plurality of components before the integration. According to various embodiments, the operations performed by the modules, programs, or other components may be executed sequentially, in parallel, repeatedly, or heuristically, or one or more of the operations may be executed in a different order or omitted, or one or more other operations may be added.

### DESCRIPTION OF SYMBOLS

10: System
100: Server
110: Memory
120: Processor
130: Input/Output interface
140: Communication interface
150: Counseling center
160: Enhancement center
170: Information center
200: Client terminal
300: Counselor terminal

1. An operation method performed by a server operated by a processor, the method comprising:
an operation of acquiring basic information related to a client;
a counseling center operation of providing a virtual reality space where the client may conduct psychological counseling on the basis of the basic information, allowing the client to conduct the psychological counseling, and recommending a first digital therapeutic among a plurality of digital therapeutics including an alleviation program for alleviating psychological risks of the client by analyzing a result of the psychological counseling;
an information center operation of extracting customized information on the client on the basis of at least one among the result of the psychological counseling and the first digital therapeutic, and providing medical information including the extracted customized information; and
an enhancement center operation of allowing the client to perform at least one among the first digital therapeutic and a second digital therapeutic selected by the client among the plurality of digital therapeutics, recording a result of performing at least one among the first digital therapeutic and the second digital therapeutic, and changing and storing the customized information on the basis of the recorded result of performing the digital therapeutic.

2. The method according to claim 1, wherein the information center operation further includes an operation of performing a cognitive and emotional test on the basis of at least one among questionnaire information, behavioral information, and biometric information, wherein the questionnaire information includes information on a result of answering a predetermined questionnaire by the client, the behavioral information includes information on a response time and an error rate measured by the client by executing a predetermined measurement program, and the biometric information includes information on nonverbal elements, psychological index elements, and biometric signals of the client.

3. The method according to claim 2, further comprising, after the operation of acquiring basic information and before the counseling center operation, an operation of determining whether a result of the cognitive and emotional test previously performed by the counselor exists, wherein on the basis of the determining operation, the counseling center operation is performed when a result of the cognitive and emotional test exists, and the counseling center operation is performed subsequently after the cognitive and emotional test is performed when a result of the cognitive and emotional test does not exist.

4. The method according to claim 2, wherein the information center operation performs an operation of allowing the counselor to perform the cognitive and emotional test after the counseling center operation, and the medical information further includes information on a result of the cognitive and emotional test.

5. The method according to claim 1, wherein the first digital therapeutic among the plurality of digital therapeutics including the alleviation program includes: at least one among a counselor-recommended digital therapeutic recommended by the counselor by connecting the client to one of a plurality of counselors in the virtual reality space and performing psychological counseling by the counselor; and an AI-recommended digital therapeutic recommended by artificial intelligence on the basis of information acquired in the psychological counseling process performed by the counselor.

6. The method according to claim 1, wherein the medical information further includes academic information updated through medical seminars.

7. The method according to claim 6, wherein the academic information is independently updated whenever the medical seminars are held or at predetermined intervals.

8. The method according to claim 7, wherein when the academic information is updated, the customized information of each client is collectively changed on the basis of the updated academic information.

9. The method according to claim 1, wherein the customized information further includes information related to precautions needed for supporting the client, which is provided to a guardian of the client.

10. The method according to claim 1, wherein the enhancement center operation further includes an operation of providing a digital therapeutic for an element related to the client, among elements including at least one among post-traumatic stress disorder, personality disorder, sexual disorder, eating disorder, sleep disorder, adjustment disorder, and impulse control disorder, as the second digital therapeutic.

11. The method according to claim 1, wherein the enhancement center operation further includes an operation of providing a digital therapeutic for an element related to the client, among elements including at least one among musculoskeletal system, dry eye, tinnitus, chronic fatigue, decreased vision, and chest pain, as the second digital therapeutic.

12. The method according to claim 1, wherein the enhancement center operation further includes an operation of providing a digital therapeutic for an element related to the client, among elements including at least one among stroke, smoking, obesity, alcoholism, migraine, and cancer, as the second digital therapeutic.

13. A server comprising:
a memory including instructions; and
a processor that performs a predetermined operation based on the instructions, wherein
the operation of the processor includes:
an operation of acquiring basic information related to a client;
a counseling center operation of providing a virtual reality space where the client may conduct psychological counseling on the basis of the basic information, allowing the client to conduct the psychological counseling, and recommending a first digital therapeutic among a plurality of digital therapeutics including an alleviation program for alleviating psychological risks of the client by analyzing a result of the psychological counseling;
an information center operation of extracting customized information on the client on the basis of at least one among the result of the psychological counseling and the first digital therapeutic, and providing medical information including the extracted customized information; and
an enhancement center operation of allowing the client to perform at least one among the first digital therapeutic and a second digital therapeutic selected by the client among the plurality of digital therapeutics, recording a result of performing at least one among the first digital therapeutic and the second digital therapeutic, and changing and storing the customized information on the basis of the recorded result of performing the digital therapeutic.
